(19) United States
(12) Patent Application Publication
(10) Pub. No.: US 2005/0008704 A1
(43) Pub. Date: Jan. 13, 2005

(54) Pharmaceutical composition for solubility enhancement of hydrophobic drugs

(76) Inventors: Anup Kumar Ray, Staten Island, NY (US); Indranil Nandi, Plainsboro, NJ (US); Suresh Palaniswamy, East Windsor, NJ (US); Pablo Davila, East Windsor, NJ (US); Aakanksha Harshad Vora, Dayton, NJ (US)

Correspondence Address: NOVARTIS;CORPORATE INTELLECTUAL PROPERTY, ONE HEALTH PLAZA 430/2, EAST HANOVER, NJ, 07936-1080, US

(21) Appl. No.: 10/618,545

(22) Filed: Jul. 11, 2003

(51) Int. Cl. A61K 31/704; A61K 31/56; A61K 31/5513; A61K 31/58

(52) U.S. Cl. 424/486; 514/171; 514/649; 514/35; 514/221; 514/571; 514/616; 514/174

(57) ABSTRACT

The present invention provides a pharmaceutical composition having enhanced solubility comprising a drug and polyethylene glycol, wherein the ratio of polyethylene glycol to drug by weight is from about 0.2:1 to about 10:1, and the polyethylene glycol has a melting point of at least 37° C. The pharmaceutical compositions exhibit rapid dissolution upon contact with physiological solvents, such as water, saliva or gastrointestinal fluids.

### FIELD OF THE INVENTION

The present invention provides a pharmaceutical composition having enhanced solubility comprising a drug and polyethylene glycol, wherein the ratio of polyethylene glycol to drug by weight is from about 0.2:1 to about 10:1, and the polyethylene glycol has a melting point of at least 37° C.

### BACKGROUND OF THE INVENTION

Hydrophobic drugs, i.e., drugs having poor solubility in aqueous solution, present difficult formulation problems for effective administration to patients. A well-designed formulation must, at a minimum, be capable of presenting a therapeutically effective amount of the hydrophobic drug to the desired absorption site, in an absorbable form. Even this minimal functionality is difficult to achieve with hydrophobic drugs because of the slow disintegration or dissolution. Especially in intestinal fluid, a drug that does not dissolve sufficiently cannot pass via the intestinal wall membrane into the bloodstream, and is simply excreted by the individual via their intestinal tract without providing a therapeutic benefit.

Moreover, when such poorly soluble drugs are formed into tablets, the process used to prepare the tablets may further reduce the disintegrating or dissolving properties of such drugs. A tableting process generally requires high compression of pharmaceutical ingredients which hinders the disintegration and wetting of the interior portion of the tablet which reduces the disintegrating or dissolving properties of the tablet. Thus, to increase the dissolution rate, tablets are commonly formulated with relatively large amounts of disintegrant and carrier materials. However, increasing the amount of disintegrant and carrier material deleteriously effects either the size of the tablet or the drug loading of the tablet.

U.S. Pat. Nos. 5,811,120 and 5,972,383 describe pharmaceutical formulations containing a hydrophobic drug, raloxifene hydrochloride and a surfactant selected from a sorbitan fatty acid ester or a polyoxyethylene sorbitan fatty acid ester, polyvinylpyrrolidone and a water-soluble diluent selected from a polyol or sugar.

It would be desirable to develop a pharmaceutical composition having enhanced solubility, especially for hydrophobic drugs. In addition, the pharmaceutical composition should be suitable for tablet formulations.

### SUMMARY OF THE INVENTION

The invention provides a pharmaceutical composition having enhanced solubility comprising a drug and polyethylene glycol, wherein the ratio of polyethylene glycol to drug by weight is from about 0.2:1 to about 10:1, and the polyethylene glycol has a melting point of at least 37° C.

According to another aspect, the invention provides a tablet having enhanced solubility comprising a hydrophobic drug and polyethylene glycol, wherein the ratio of polyethylene glycol to drug by weight is from about 0.2:1 to about 10:1, and the polyethylene glycol has a melting point of at least 37° C.

According to another aspect, the invention provides a method of preparing a pharmaceutical composition having enhanced solubility comprising a drug and polyethylene glycol, wherein the ratio of polyethylene glycol to drug by weight is from about 0.2:1 to about 10:1, and the polyethylene glycol has a melting point of at least 37° C., said method comprising:
(a) combining polyethylene glycol with a drug and optionally one or more excipients to form a premix;
(b) adding a solvent and optionally a surfactant to the premix formed in Step (a) to form a wet granulation; and
(c) drying the wet granulation to form a pharmaceutical composition which is encapsulated or tableted.

According to another aspect, the invention provides a method of preparing a pharmaceutical composition having enhanced solubility comprising a drug and polyethylene glycol, wherein the ratio of polyethylene glycol to drug by weight is from about 0.2:1 to about 10:1, and the polyethylene glycol has a melting point of at least 37° C., said method comprising:
(a′) combining a drug and optionally one or more excipients to form a premix;
(b′) adding a mixture comprising a solvent and polyethylene glycol to the premix formed in Step (a′) to form a wet granulation; and

(c′) drying the wet granulation to form a pharmaceutical composition which is encapsulated or tableted.

According to another aspect, the invention provides a method of preparing a pharmaceutical composition having enhanced solubility comprising a drug and polyethylene glycol, wherein the ratio of polyethylene glycol to drug by weight is from about 0.2:1 to about 10:1, and the polyethylene glycol has a melting point of at least 37° C., said method comprising:
(a″) combining a drug with melted polyethylene glycol and optionally a surfactant to form a slurry; and
(b″) cooling the slurry formed in Step (a″) to form a solid;
(c″) milling the solid formed in Step (b″) to form granules, and
(d″) mixing at least one excipient with the granules to form a pharmaceutical composition which is encapsulated or tableted.

The pharmaceutical compositions having enhanced solubility of the invention exhibit rapid dissolution upon contact with physiological solvents, such as water, saliva or gastrointestinal fluids, due to the presence of a critical type and amount of polyethylene glycol, as compared to pharmaceutical compositions which do not contain such polyethylene glycol.

### BRIEF DESCRIPTION OF THE DRAWINGS

FIG. 1 is a dissolution profile of five anagrelide samples.

FIG. 2 is a dissolution profile of three modafinil samples.

FIG. 3 is a dissolution profile of five raloxifene samples.

FIG. 4 is a dissolution profile of five raloxifene samples.

### DESCRIPTION OF THE INVENTION

The pharmaceutical compositions of the invention comprise a drug, preferably a hydrophobic drug, and polyethylene glycol (PEG). Examples of hydrophobic drugs include, but are not limited to, raloxifene, paroxetine, glimepiride, anagrelide, modafinil, paroxetine, cabergoline, replaginide, glipizide, benzodiazepines, clofibrate, chlorpheniramine, dinitirate, digoxin, digitoxin, ergotamin tartate, estradiol, fenofibrate, griseofulvin, hydrochlorothiazide, hydrocortisone, isosorbide, medrogeston, oxyphenbutazone, prednisolone, prednisone, polythiazide, progensterone, spironolactone, tolbutamide, 10,11-dihydro-5H-dibenzo[a, d]cyclo-heptene-5-carboxamide; 5H-dibenzo[a, d]cycloheptene-5-carboxamide, fish oil and the like, including pharmaceutical acceptable salts thereof. Preferably, the hydrophobic drug is selected from raloxifene, paroxetine, glimepiride, anagrelide and modafinil, including pharmaceutical acceptable salts thereof. A combination of drugs may also be used. While the invention is illustrated with particularly hydrophobic drugs, the pharmaceutical composition of the invention is also applicable to more soluble drugs in need of enhanced dissolution and bioavailability.

The term “pharmaceutically acceptable salt” refers to those salts of the above described drugs that are not substantially toxic at the dosage administered to achieve the desired effect and do not independently possess significant pharmacological activity. The salts included within the scope of this term are pharmaceutically acceptable acid addition salts of a suitable inorganic or organic acid. Suitable inorganic acids are, e.g., hydrochloric, hydrobromic, sulfuric and phosphoric acids. Suitable organic acids include carboxylic acids, such as acetic, propionic, glycolic, lactic, pyruvic, malonic, succinic, fumaric, malic, tartaric, citric, cyclamic, ascorbic, maleic, hydroxymaleic, dihydroxymaleic, benzoic, phenylacetic, 4-aminobenzoic, 4-hydroxybenzoic, anthranillic, cinnamic, salicylic, 4-aminosalicyclic, 2-phenoxybenzoic, 2-acetoxybenzoic and mandelic acid; sulfonic acids, such as methanesulfonic, ethanesulfonic and β-hydroxyethanesulfonic acid. In addition, “pharmaceutically acceptable salts” include those salts of the above described drugs formed with inorganic and organic bases, such as those of alkali metals, e.g., sodium, potassium and lithium; alkaline earth metals, e.g., calcium and magnesium; light metals of group IIIA, e.g., aluminum; organic amines, e.g., primary, secondary or tertiary amines, such as cyclohexylamine, ethylamine, pyridine, methylaminoethanol and piperazine. The salts are prepared by conventional means by one of ordinary skill in the art as, e.g., by treating a compound with an appropriate acid or base. Such salts can exist in either a hydrated or substantially anhydrous form.

Preferably, the pharmaceutically acceptable salt of raloxifene is raloxifene hydrochloride. Preferably, the pharmaceutically acceptable salt of paroxetine is paroxetine hydrochloride. Preferably, the pharmaceutically acceptable salt of glimepiride is glimepiride hydrochloride. Preferably, the pharmaceutically acceptable salt of anagrelide is anagrelide hydrochloride.

The amount of drug in the pharmaceutical compositions is preferably from about 20 mg to about 2000 mg. More preferably, the amount of drug in the pharmaceutical compositions is from about 60 mg to about 200 mg.

Polyethylene glycol is a condensation polymer of ethylene glycol having the formula HOCH₂(CH₂OCH₂)ₙCH₂OH, wherein n is the average number of oxyethylene groups. Preferably, n is from 20-204. The PEG should have a m.p. of at least about 37° C. In addition, the PEG preferably has an average molecular weight (m.w.) from about 950 to about 20,000, more preferably from about 2700 to about 9000. A combination of PEGs may also be used. Thus, grades of PEG 1000 and upwards are suitable for use in the present invention. The average m.w. and m.p. of preferred PEGs are typically as follows: PEG 1000: m.w. 950-1050, m.p. 37-40° C.; PEG 1500: m.w. 1400-1600, m.p. 44-48° C.; PEG 1540: m.w. 1300-1600, m.p. 40-48° C.; PEG 2000: m.w. 1800-2200, m.p. 45-50° C.; PEG 3000: m.w. 2700-3300, m.p. 48-54° C.; PEG 4000: m.w. 3000-4800, m.p. 50-58° C.; PEG 6000: m.w. 5400-6600, m.p. 55-63° C.; PEG 8000: m.w. 7000-9000, m.p. 60-63° C.; and PEG 20000: m.w. 15000-20000, m.p. 60-63° C.

The ratio of polyethylene glycol to drug by weight is from about 0.2:1 to about 10:1. Preferably, the ratio of polyethylene glycol to drug by weight is from about 0.5:1 to about 5:1. More preferably the ratio of polyethylene glycol to drug by weight is from about 0.7:1 to about 2:1, most preferably the ratio is 1:1.

The pharmaceutical compositions of the invention may additionally include a surfactant or a combination of surfactants. Preferred surfactants include: polyoxyethylene-sorbitan-fatty acid esters, also called polysorbates, e.g., mono- and tri-lauryl, palmityl, stearyl and oleyl esters of the type known and commercially-available under the trademark TWEEN including the following products:
Tween 20 [polyoxyethylene(20)sorbitanmonolaurate]
Tween 21 [polyoxyethylene(4)sorbitanmonolaurate]
Tween 40 [polyoxyethylene(20)sorbitanmonopalmitate]
Tween 60 [polyoxyethylene(20)sorbitanmonostearate]
Tween 65 [polyoxyethylene(20)sorbitantristearate]
Tween 80 [polyoxyethylene(20)sorbitanmonooleate]
Tween 81 [polyoxyethylene(5)sorbitanmonooleate]
Tween 85 [polyoxyethylene(20)sorbitantrioleate]

More preferably, the surfactant is TWEEN 80 [polyoxyethylene(20)sorbitanmonooleate].

The surfactant is preferably present in an amount of from about 0.01 weight percent (wt %) to about 20 wt %, based on the total weight of the pharmaceutical composition. More preferably, the surfactant is present in an amount of from about 1 wt % to about 5 wt %, based on the total weight of the composition.

It is within the scope of the invention for the pharmaceutical compositions, in addition to the hydrophobic drug, PEG and optionally a surfactant, to include one or more pharmaceutically acceptable excipients. Examples of such excipients are enteric-coating agents, diluents, binders, anti-caking agents, amino acids, fibers, solubilizers, disintegrants, fillers, lubricants, emulsifiers, flavorants, solvents, buffers, stabilizers, colorants, dyes, anti-oxidants, anti-adherents, preservatives, electrolytes, glidants and carrier materials. A combination of excipients may also be used. Such excipients are known to those skilled in the art, and thus, only a limited number will be specifically referenced.

Examples of fillers include lactose anhydrous, microcrystalline cellulose, starch, pregelatinized starch, modified starch, dibasic calcium phosphate dihydrate, calcium sulfate trihydrate, calcium sulfate dihydrate, calcium carbonate, lactose, dextrose, sucrose, mannitol and sorbitol. A combination of fillers may also be used. Preferred fillers are mannitol and lactose monohydrate.

Examples of solvents include water, acetonitrile, ethyl acetate, acetone, benzene, toluene, dioxane, dimethylformamide, chloroform, methylene chloride, ethylene chloride, carbon tetrachloride, chlorobenzene, acetone, methanol, ethanol, isopropanol and butanol. A combination of solvents may also be used. Preferably, the solvent is water.

Examples of lubricants include magnesium stearate, calcium stearate, zinc stearate, talc, propylene glycol, PEG, stearic acid, vegetable oil, sodium benzoate, sodium lauryl sulfate, magnesium lauryl sulfate, mineral oil and polyoxyethylene monostearate. A combination of lubricants may also be used. A preferred lubricant is magnesium stearate.

Examples of enteric-coating agents include hydroxypropylmethylcellulose phthalate, methacrylic acid-methacrylic acid copolymer, methyl methacrylate-methacrylic acid copolymer, polyvinyl acetate-phthalate and cellulose acetate phthalate.

Examples of binders include starches, e.g., potato starch, wheat starch, corn starch; gums, such as gum tragacanth, acacia gum and gelatin; microcrystalline cellulose, e.g., products known under the registered trademarks Avicel, Filtrak, Heweten or Pharmacel, hydroxypropyl cellulose, hydroxyethyl cellulose and hydroxypropylmethyl cellulose; and polyvinyl pyrrolidone, e.g., Povidone.

Examples of glidants include silica, magnesium trisilicate, powdered cellulose, starch, talc and tribasic calcium phosphate. Colloidal silica, e.g., Aerosil, is particularly preferred.

Examples of solubilizers and/or emulsifiers include sorbitan fatty acid esters, such as sorbitan trioleate; phosphatides, such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20.

Examples of disintegrants include:
(i) natural starches, such as maize starch, potato starch and the like, directly compressible starches, e.g., Sta-rx® 1500; modified starches, e.g., carboxymethyl starches and sodium starch glycolate, available as Primojel®, Explotab®, Explosol®;
and starch derivatives, such as amylose;
(ii) cross-linked polyvinylpyrrolidones, e.g., crospovidones, such as Polyplasdonee XL and Kollidon® CL;
(iii) alginic acid and sodium alginate;
(iv) methacrylic acid-divinylbenzene co-polymer salts, e.g., Amberlite® IRP-88; and
(v) cross-linked sodium carboxymethylcellulose, available as, e.g., Ac-di-sol®, Primellose®, Pharmacel® XL, Explocel® and Nymcel® ZSX. Additional disintegrants also include hydroxypropyl cellulose, hydroxypropylmethyl cellulose, croscarmellose sodium, sodium starch glycolate, polacrillin potassium, polyacrylates, such as Carbopol®, magnesium aluminium silicate and bentonite.

Examples of carrier materials include cross-linked polyvinyl pyrrolidone, carboxymethylamide, potassium methacrylatedivi nylbenzene co-polymer, high-molecular weight polyvinylacohols, low-molecular weight polyvinylalcohols, medium-viscosity polyvinylalcohols, polyoxyethyleneglycols, non-cross-linked polyvinylpyrrolidone, PEG, sodium alginate, galactomannone, carboxypolymethylene, sodium carboxymethyl starch, sodium carboxymethyl cellulose or microcrystalline cellulose; polymerizates, as well as co-polymerizates of acrylic acid and/or methacrylic acid and/or their esters, such as, but not limited to, poly(methyl methacrylate), poly(ethyl methacrylate), poly(butyl methacylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) or poly(octadecyl acrylate); co-polymerizates of acrylic and methacrylic acid esters with a lower ammonium group content, e.g., Eudragit™ RS, available from Rohm; co-polymerizates of acrylic and methacrylic acid esters and trimethyl ammonium methacrylate, e.g., Eudragit™ RL, available from Rohm; polyvinyl acetate; fats, oils, waxes, fatty alcohols; hydroxypropyl methyl cellulose phthalate or acetate succinate; cellulose acetate phthalate, starch acetate phthalate, as well as polyvinyl acetate phthalate, carboxy methyl cellulose; methyl cellulose phthalate, methyl cellulose succinate, -phthalate succinate, as well as methyl cellulose phthalic acid half ester; zein; ethyl cellulose, as well as ethyl cellulose succinate; shellac, gluten; ethylcarboxyethyl cellulose; ethylacrylate-maleic acid anhydride co-polymer; maleic acid anhydride-vinyl methyl ether co-polymer; styrol-maleic acid co-polymerizate; 2-ethyl-hexyl-acrylate maleic acid anhydride; crotonic acid-vinyl acetate co-polymer; glutaminic acid/glutamic acid ester co-polymer; carboxymethylethylcellulose glycerol monooctanoate; cellulose acetate succinate; polyarginine; poly(ethylene), poly(ethylene) low-density, poly(ethylene) high-density, poly(propylene), poly(ethylene oxide), poly(ethylene terephthalate), poly(vinyl isobutyl ether), poly(vinyl chloride) or polyurethane.

In one embodiment of the invention, the pharmaceutical composition of the invention is prepared by a process comprising:
(a) combining polyethylene glycol with a drug and optionally one or more excipients to form a premix;
(b) adding a solvent and optionally a surfactant to the premix formed in Step (a) to form a wet granulation;
(c) drying the wet granulation to form dried granules, and optionally milling the dried granules; and
(d) optionally mixing at least one excipient with the granules to form a pharmaceutical composition which is encapsulated or tableted.

In another embodiment of the invention, the pharmaceutical composition of the invention is prepared by a process comprising:
(a′) combining a drug and optionally one or more excipients to form a premix;
(b′) adding a mixture comprising a solvent and polyethylene glycol to the premix formed in Step (a′) to form a wet granulation;
(c′) drying the wet granulation to form dried granules, and optionally milling the dried granules; and
(d′) optionally mixing at least one excipient with the granules to form a pharmaceutical composition which is encapsulated or tableted.

In an additional embodiment of the invention, the pharmaceutical composition of the invention is prepared by a process comprising:
(a″) combining a drug with melted polyethylene glycol and optionally a surfactant to form a slurry; and
(b″) cooling the slurry formed in Step (a″) to form a solid;
(c″) milling the solid formed in Step (b″) to form granules, and (d″) mixing at least one excipient with the granules to form a pharmaceutical composition which is encapsulated or tableted.

Drying techniques useful for drying the granulation include spray-drying, flash drying, ring drying, micron drying, tray drying, vacuum drying, radio-frequency drying, microwave drying, and lyophilizing.

The pharmaceutical compositions of the invention may be in the form of a capsule, caplet, powder, disc or tablet. In a preferred embodiment, the pharmaceutical compositions are in the form of a tablet.

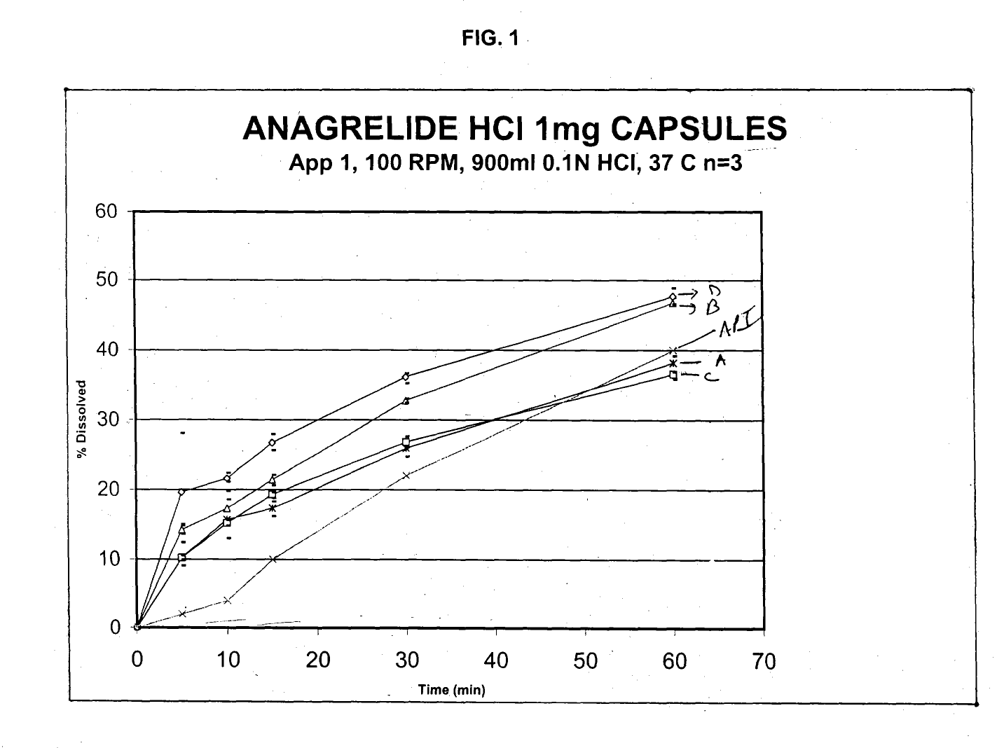

Referring to the drawings, FIG. 1 is a graph illustrating the average dissolved anagrelide during a period of 70 minutes from five different samples containing anagrelide. A USP Apparatus I dissolution apparatus was used at 100 rpm which containing 900 mL of 0.1 N HCL at 37° C. Each sample was tested three times and the average was plotted:
Sample A contained 0.5 mg of PEG 4500, 1 mg of anagrelide and 0.03 mg of polysorbate 80.
Sample B contained 1 mg of PEG 4500, 1 mg of anagrelide and 0.04 mg of polysorbate 80.
Sample C contained 0.5 mg of PEG 4500 and 1 mg of anagrelide.
Sample D contained 1 mg of PEG 4500 and 1 mg of anagrelide.
Sample E contained 1 mg of anagrelide.

FIG. 1 clearly shows that a 1:1 ratio of PEG 4500 to anagrelide increases the solubility of anagrelide with or without the presence of a surfactant. Sample D which contained a 1:1 ratio of PEG 4500 to anagrelide without a surfactant dissolved faster than Sample B which contained a 1:1 ratio of PEG 4500 to anagrelide and a surfactant.

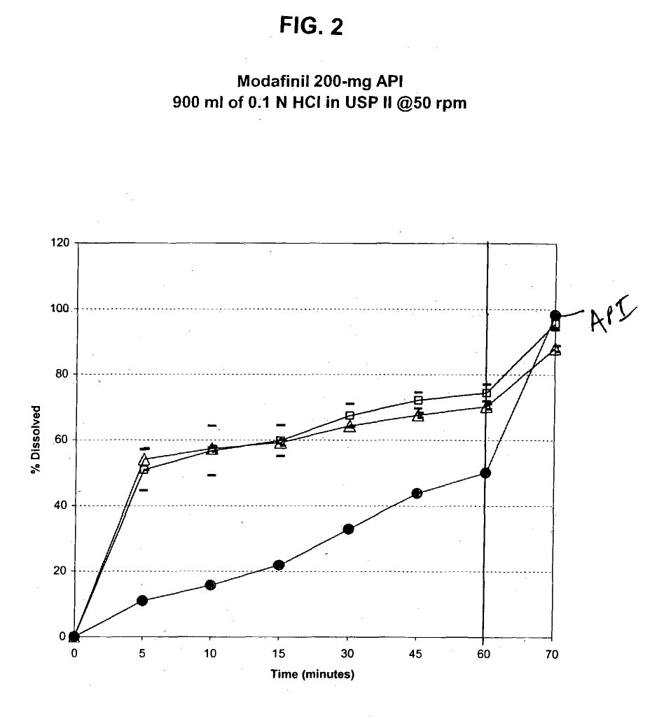

Referring to the drawings, FIG. 2 is a graph illustrating the average dissolved modafinil during a period of 70 minutes from two different samples containing modafinil. A USP Apparatus II dissolution apparatus was used at 50 rpm containing 900 mL of 0.1 N HCL at 37° C. Each sample was tested three times and the average was plotted.
Sample A contained 200 mg of PEG 4500 and 200 mg of modafinil.
Sample B contained 200 mg of PEG 3350 and 200 mg of modafinil.
Sample C contained 200 mg of modafinil.

FIG. 2 clearly shows that different PEG's can be used to increase the solubility of hydrophilic drugs provided the PEG is a solid at room temperature (about 25° C.). In addition, FIG. 2 shows that the presence of PEG 4500 and PEG 3350 significantly increases the dissolution or solubility of modafinil.

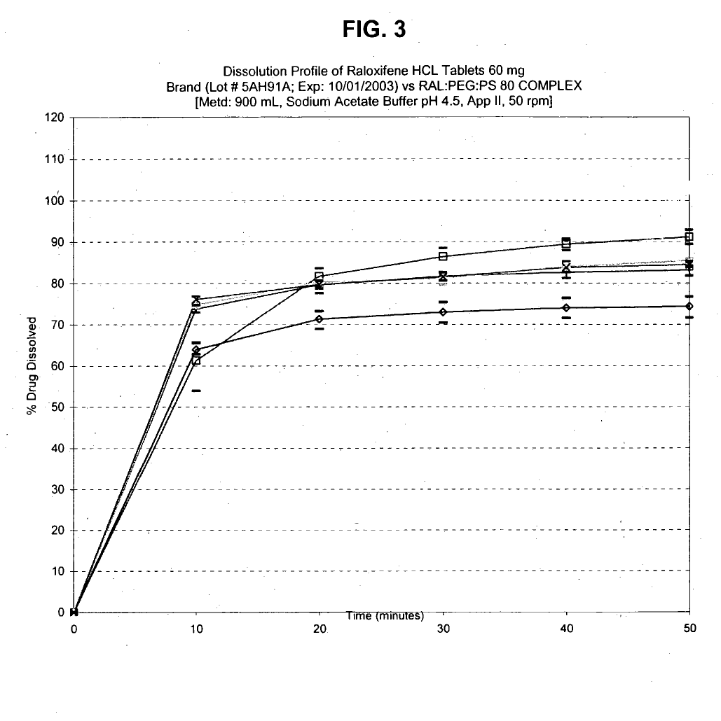

Referring to the drawings, FIG. 3 is a graph illustrating the average dissolved raloxifene during a period of 50 minutes from four different samples containing raloxifene. A USP Apparatus II dissolution apparatus was used at 50 rpm which containing 900 mL of sodium acetate buffer pH 4.5, at 37° C. Each sample was tested three times and the average was plotted. The only difference in the samples was the amount of PEG 4500.
Sample A contained 12 mg of PEG 4500, 60 mg of raloxifene and 7.2 mg of polysorbate 80.
Sample B contained 30 mg of PEG 4500, 60 mg of raloxifene and 7.2 mg of polysorbate 80.
Sample C contained 60 mg of PEG 4500, 60 mg of raloxifene and 7.2 mg of polysorbate 80.
Sample D contained 120 mg of PEG 4500, 60 mg of raloxifene and 7.2 mg of polysorbate 80.

FIG. 3 clearly shows that when the ratio of PEG 4500 to raloxifene by weight is from 0.5:1 to 2:1, the solubility of raloxifene is significantly increased.

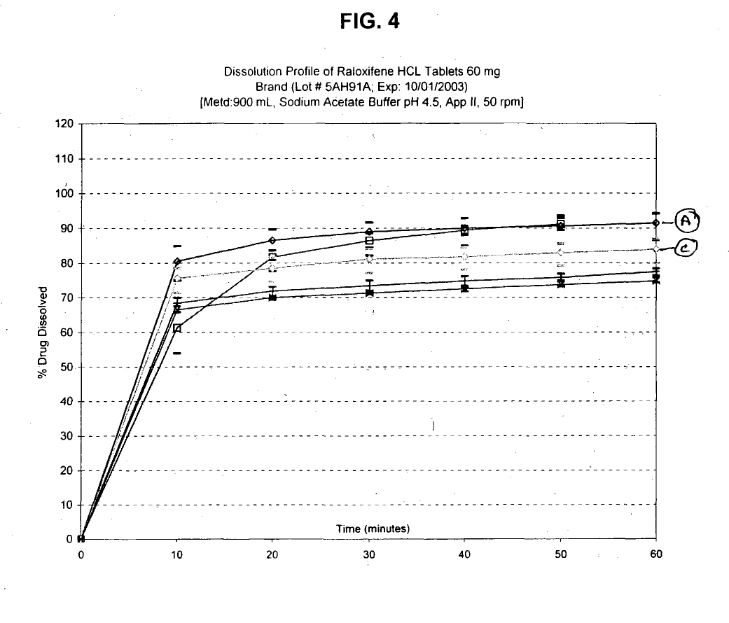

Referring to the drawings, FIG. 4 is a graph illustrating the average dissolved raloxifene during a period of 60 minutes from three different samples containing raloxifene. A USP Apparatus II dissolution apparatus was used at 50 rpm which containing 900 mL of sodium acetate buffer pH 4.5, at 37° C. Each sample was tested three times and the average was plotted.
Sample A contained 60 mg of PEG 4500, 60 mg of raloxifene and 7.2 mg of polysorbate 80.
Sample B contained 60 mg of PEG 4500 and 60 mg of raloxifene.
Sample C contained 60 mg of PEG 8000, 60 mg of raloxifene, and 7.2 mg of polyoxyethylene-polyoxypropylene copolymer (Poloxamer 188).
Sample D contained 60 mg of raloxifene and other excipients.
Sample E contained 60 mg of raloxifene.

FIG. 4 clearly shows that the solubility of raloxifene is increased in the presence of a surfactant, provided that a polyethylene glycol is also used.

The following non-limiting examples illustrate further aspects of the invention.

### EXAMPLE 1

Preparation of Raloxifene HCl-PEG Solid Dispersion with Surfactant.

PEG 4500, 2.5 g, was placed in a 50 mL beaker with a magnetic stirrer and melted to liquid over hot plate. Polysorbate 80, 5 drops (about 2%) was added to the beaker and mixed. The mixture was stirred vigorously and to this mixture was added 2.5 g of raloxifene HCl to form a dispersion. A uniform mixing was done at room temperature before cooling the mixture. The solid obtained was milled and dried overnight under vacuum at room temperature.

### Example 2

Preparation of Raloxifene HCl-PEG Solid Dispersion with Surfactant.

The procedure set forth in Example 1 was followed except that PEG 4500 was replaced with PEG 8000 and the amount of PEG 8000 to Raloxifene HCl was varied from 0.2:1 to 5:1 and the amount of polysorbate 80 varied from 1-5%.

### EXAMPLE 3

Preparation of Raloxifene HCl-PEG Solid Dispersion without Surfactant.

PEG 4500, 2.5 g, was placed in a 50 mL beaker with a magnetic stirrer and melted to liquid over hot plate. Isopropyl alcohol, 5 mL, was added to the beaker and mixed. The mixture was stirred vigorously and to it was dispersed 2.5 g raloxifene HCl. A uniform mixing was done at room temperature before cooling the mixture. The solid obtained was milled, and dried overnight under vacuum at room temperature.

### EXAMPLE 4

Preparation of Paroxetine HCl-PEG Solid Dispersion with Surfactant.

PEG 4500, 2.5 g, was placed in a 50 mL beaker with a magnetic stirrer and melted to liquid over hot plate. Polysorbate 80, 5 drops (about 2%) was added to the beaker and mixed. The mixture was stirred vigorously and to it was dispersed 2.5 g paroxetine HCl. A uniform mixing was done at room temperature to cool the mixture. The solid obtained was milled, and dried overnight under vacuum at room temperature.

### EXAMPLE 5

Preparation of Paroxetine HCl-PEG Solid Dispersion with Surfactant.

The procedure set forth in Example 4 was followed except that PEG 4500 was replaced with PEG 8000 and the amount of PEG 8000 to paroxetine HCl was varied from 0.2:1 to 5:1 and the amount of polysorbate 80 varied from 1-5%.

### EXAMPLE 6

Preparation of Paroxetine HCl-PEG Solid Dispersion without Surfactant.

PEG 4500, 2.5 g, was placed in a 50 mL beaker with a magnetic stirrer and melted to liquid over hot plate. Isopropyl alcohol, 5 mL, was added to the beaker and mixed. The mixture was stirred vigorously and 2.5 g paroxetine HCl was added. A uniform mixing was done at room temperature to cool the mixture. The solid obtained was milled, and dried overnight under vacuum at room temperature.

### EXAMPLE 7

Preparation of Glimepiride HCl-PEG Solid Dispersion with Surfactant.

PEG 4500, 2.5 g, was placed in a 50 mL beaker with a magnetic stirrer and melted to liquid over hot plate. Polysorbate 80, 5 drops (about 2%) was added to the beaker and mixed. The mixture was stirred vigorously and 2.5 g glimepiride HCl was added. A uniform mixing was done at room temperature to cool the mixture. The solid obtained was milled, and dried overnight under vacuum at room temperature.

### EXAMPLE 8

Preparation of Glimepiride HCl-PEG Solid Dispersion with Surfactant.

The procedure set forth in Example 7 was followed except that PEG 4500 was replaced with PEG 8000 and the amount of PEG 8000 to glimepiride HCl was varied from 0.2:1 to 5:1 and the amount of polysorbate 80 varied from 1-5%.

### EXAMPLE 9

Preparation of Glimepiride HCl-PEG Solid Dispersion without Surfactant.

PEG 4500, 2.5 g, was placed in a 50 mL beaker with a magnetic stirrer and melted to liquid over hot plate. Isopropyl alcohol, 5 mL, was added to the beaker and mixed. The mixture was stirred vigorously and 2.5 g glimepiride HCl was added. A uniform mixing was done at room temperature to cool the mixture. The solid obtained was milled, and dried overnight under vacuum at room temperature.

### EXAMPLE 10

Preparation of Anagrelide HCl Monohydrate-PEG Solid Dispersion with Surfactant.

PEG 4500, 2.5 g, was placed in a 50 mL beaker with a magnetic stirrer and melted to liquid over hot plate. Polysorbate 80, 5 drops (about 2%) was added to the beaker and mixed. The mixture was stirred vigorously and 2.5 g anagrelide HCl monohydrate was added. A uniform mixing was done at room temperature to cool the mixture. The solid obtained was milled, and dried overnight under vacuum at room temperature.

### EXAMPLE 11

Preparation of Anagrelide HCl Monohydrate-PEG Solid Dispersion with Surfactant.

The procedure set forth in Example 10 was followed except that PEG 4500 was replaced with PEG 8000 and the amount of PEG 8000 to anagrelide HCl monohydrate was varied from 0.2:1 to 5:1 and the amount of polysorbate 80 varied from 1-5%.

### EXAMPLE 12

Preparation of Anagrelide HCl Monohydrate-PEG Solid Dispersion without Surfactant.

PEG 4500, 2.5 g, was placed in a 50 mL beaker with a magnetic stirrer and melted to liquid over hot plate. Isopropyl alcohol, 5 mL, was added to the beaker and mixed. The mixture was stirred vigorously and 2.5 g anagrelide HCl monohydrate was added. A uniform mixing was done at room temperature to cool the mixture. The solid obtained was milled, and dried overnight under vacuum at room temperature.

### EXAMPLE 13

Preparation of Modafinil-PEG Solid Dispersion with Surfactant.

PEG 4500, 2.5 g, was placed in a 50 mL beaker with a magnetic stirrer and melted to liquid over hot plate. Polysorbate 80, 5 drops (about 2%) was added to the beaker and mixed. The mixture was stirred vigorously and 2.5 g modafinil was added. A uniform mixing was done at room temperature to cool the mixture. The solid obtained was milled, and dried overnight under vacuum at room temperature.

### EXAMPLE 14

Preparation of Modafinil-PEG Solid Dispersion without Surfactant.

PEG 4500, 2.5 g, was placed in a 50 mL beaker with a magnetic stirrer and melted to liquid over hot plate. Isopropyl alcohol, 5 mL, was added to the beaker and mixed. The mixture was stirred vigorously and 2.5 g modafinil was added. A uniform mixing was done at room temperature to cool the mixture. The solid obtained was milled, and dried overnight under vacuum at room temperature.

### EXAMPLE 15

Preparation of Raloxifene Tablet Composition.

| Item # | Ingredients | mg/unit | % |
|---|---|---|---|
| 1 | Raloxifene HCl | 60 | 23.62 |
| 2 | Lactose Anhydrous | 120 | 47.24 |
| 3 | Lactose Hydrous | 30 | 11.81 |
| 4 | PEG 4500 | 26 | 10.24 |
| 5 | Polysorbate 80 | 2.4 | 0.94 |
| 6 | Crospovidone | 6 | 2.36 |
| 7 | Purified Water | q.s. | — |
| 8 | Crospovidone | 8.4 | 3.31 |
| 9 | Magnesium Stearate | 1.2 | 0.47 |
| Total | | 254 | 100 |

The tablet composition was prepared by weighing items 1-6. The PEG 4500 was crushed and added to a mixture of raloxifene, lactose anhydrous and lactose hydrous. The crospovidone (item 6) was added to the mixture. A granulating solution containing 2.5 g of water and polysorbate 80 (Tween 80) was prepared and added to the mixture to form a wet granulation. The wet granulation was dried in an oven at 55° C. to form dried granules. The granules were sieved through a screen #20. Crospovidone (item #8) was mixed with the granules for one minute. Magnesium stearate was mixed with the granules for one minute.

### EXAMPLE 16

Preparation of Raloxifene Tablet Formulation.

The ingredients and procedure set forth in Example 15 was followed except that the PEG 4500 was mixed with the water and polysorbate 80 to form a granulating solution which was added to the premix containing raloxifene, lactose anhydrous, lactose hydrous and crospovidone (item 6).

While the invention has been described with particular reference to certain embodiments thereof, it will be understood that changes and modifications may be made by those of ordinary skill within the scope and spirit of the following claims:

1. A pharmaceutical composition having enhanced solubility comprising a drug and polyethylene glycol, wherein the ratio of polyethylene glycol to drug by weight is from about 0.2:1 to about 10:1, and the polyethylene glycol has a melting point of at least 37° C.

2. A pharmaceutical composition having enhanced solubility comprising a drug and polyethylene glycol, wherein the ratio of polyethylene glycol to drug by weight is from about 0.5:1 to about 5:1, and the polyethylene glycol has a melting point of at least 37° C.

3. A pharmaceutical composition having enhanced solubility comprising a drug and polyethylene glycol, wherein the ratio of polyethylene glycol to drug by weight is from about 0.7:1 to about 2:1, and the polyethylene glycol has a melting point of at least 37° C.

4. The composition according to claim 3, wherein the weight ratio of polyethylene glycol to drug is about 1:1.

5. The composition according to claim 1, wherein the polyethylene glycol has a melting point of at least 50° C.

6. The composition according to claim 1, wherein the drug is a hydrophobic drug.

7. The composition according to claim 6, wherein the hydrophobic drug is selected from the group consisting of raloxifene, paroxetine, glimepiride, anagrelide, modafinil, cabergoline, replaginide, glipizide, benzodiazepines, clofibrate, chlorpheniramine, dinitirate, digoxin, digitoxin, ergotamin tartate, estradiol, fenofibrate, griseofulvin, hydrochlorothiazide, hydrocortisone, isosorbide, medrogeston, oxyphenbutazone, prednisolone, prednisone, polythiazide, progensterone, spironolactone, tolbutamide, 10,11-dihydro-5H-dibenzo[a, d]cyclo-heptene-5-carboxamide; 5H-dibenzo[a,d]cycloheptene-5-carboxamide, fish oil and combinations thereof.

8. The composition according to claim 7, wherein the hydrophobic drug is selected from the group consisting of raloxifene, paroxetine, glimepiride, anagrelide and modafinil.

9. The composition according to claim 1, wherein the polyethylene glycol has the formula HOCH₂(CH₂OCH₂)ₙCH₂OH, wherein n is from 20-204.

10. The composition according to claim 9, wherein the polyethylene glycol has an average molecular weight from about 950 to about 20,000.

11. The composition according to claim 10, wherein the polyethylene glycol has an average molecular weight from about 2700 to about 9000.

12. The composition according to claim 9, wherein the polyethylene glycol is selected from the group consisting of PEG 1000, PEG 1500, PEG 1540, PEG 2000, PEG 3000, PEG 4000, PEG 4500, PEG 6000, PEG 8000 and PEG 20000.

13. The composition according to claim 1, which is essentially free of a surfactant.

14. The composition according to claim 1, which additionally comprises a surfactant.

15. The composition according to claim 14, wherein the surfactant is selected from the group consisting of reaction products of a natural or hydrogenated castor oil and ethylene oxide, polyoxyethylene-sorbitan-fatty acid esters, polyoxyethylene fatty acid esters, polyoxyethylene-polyoxypropylene co-polymers and block co-polymers, dioctylsulfosuccinate or di-[2-ethylhexyl]-succinate, phospholipids, propylene glycol mono- and di-fatty acid esters, polyoxyethylene alkyl ethers, tocopherol esters, docusate salts and combinations thereof.

16. The composition according to claim 15, wherein the surfactant is a polyoxyethylene-sorbitan-fatty acid ester.

17. The composition according to claim 16, wherein the polyoxyethylene-sorbitan-fatty acid ester is selected from the group consisting of polyoxyethylene(20)sorbitanmonolaurate, polyoxyethylene(4)sorbita nmonolaurate, polyoxyethylene(20)sorbitanmonopalmitate, polyoxyethylene(20)sorbitanmonostearate, polyoxyethylene(20)sorbitantristearate, polyoxyethylene(20)sorbitanmonooleate, polyoxyethylene(5)sorbitanmonooleate, and polyoxyethylene(20)sorbitantrioleate.

18. The composition according to claim 17, wherein the polyoxyethylene-sorbitan-fatty acid ester is polyoxyethylene(20)sorbitanmonooleate.

19. The composition according to claim 14, wherein the surfactant is present in an amount of from about 0.01 wt % to about 20 wt %, based on the total weight of the composition.

20. The composition according to claim 19, wherein the surfactant is present in an amount of from about 1 wt % to about 5 wt %, based on the total weight of the composition.

21. The composition according to claim 1, which additionally comprises at least one excipient.

22. The composition according to claim 21, wherein the excipient is selected from the group consisting of enteric coating agents, diluents, binders, anti caking agents, amino acids, fibers, solubilizers, disintegrants, fillers, lubricants, emulsifiers, flavorants, solvents, buffers, stabilizers, colorants, dyes, anti-oxidants, anti-adherents, preservatives, electrolytes, glidants, carrier materials and combinations thereof.

23. The composition according to claim 1, which is in the form selected from the group consisting of a tablet, granules, bar, block, disc, capsule, caplet and powder.

24. A method of preparing a pharmaceutical composition having enhanced solubility comprising a drug and polyethylene glycol, wherein the ratio of polyethylene glycol to drug by weight is from about 0.2:1 to about 10:1, and the polyethylene glycol has a melting point of at least 37° C., said method comprising:
(a) combining polyethylene glycol with a drug and optionally one or more excipients to form a premix;
(b) adding a solvent and optionally a surfactant to the premix formed in Step (a) to form a wet granulation; and
(c) drying the wet granulation to form a pharmaceutical composition.

25. A method of preparing a pharmaceutical composition having enhanced solubility comprising a drug and polyethylene glycol, wherein the ratio of polyethylene glycol to drug by weight is from about 0.2:1 to about 10:1, and the polyethylene glycol has a melting point of at least 37° C., said method comprising:
(a′) combining a drug and optionally one or more excipients to form a premix;
(b′) adding a mixture comprising a solvent and polyethylene glycol to the premix formed in Step (a′) to form a wet granulation; and
(c′) drying the wet granulation to form a pharmaceutical composition.

26. A method of preparing a pharmaceutical composition having enhanced solubility comprising a drug and polyethylene glycol, wherein the ratio of polyethylene glycol to drug by weight is from about 0.2:1 to about 10:1, and the polyethylene glycol has a melting point of at least 37° C., said method comprising:
(a″) combining a drug with melted polyethylene glycol and optionally a surfactant to form a slurry; and
(b″) cooling the slurry formed in Step (a″) to form a solid;
(c″) milling the solid formed in Step (b″) to form granules, and
(d″) mixing at least one excipient with the granules to form a pharmaceutical composition.
